(19) United States
(12) Plant Patent Application Publication
(10) Pub. No.: US 2016/0374245 P1
(43) Pub. Date: Dec. 22, 2016

(54) Hydrangea arborescens plant named 'NCHA3'

(50) ;

(71) Applicant: Thomas Green Ranney, Arden, NC (US)

(72) Inventor: Thomas Green Ranney, Arden, NC (US)

(73) Assignee: North Carolina State University, Raleigh, NC (US)

(21) Appl. No.: 14/545,795

(22) Filed: Jun. 18, 2015

(51) Int. Cl. A01H 5/00

(52) U.S. Cl. PLT/250

(57) ABSTRACT

A new and distinct Hydrangea cultivar named ‘NCHA3’ is disclosed, characterized by large, mop-head inflorescences with prevalent sterile flowers. Flower buds are red and open with dark pink sepals. Plant habit is compact, and plants are triploid. The new variety is a Hydrangea normally produced as an outdoor garden or container plant.

### LATIN NAME OF THE GENUS AND SPECIES

The Latin name of the novel plant variety disclosed herein is Hydrangea arborescens.

### VARIETY DENOMINATION

The seedling of Hydrangea arborescens disclosed herein has been given the varietal denomination ‘NCHA3’.

### BACKGROUND OF THE INVENTION

The present invention comprises a new and distinct hydrangea cultivar hereinafter referred to by the cultivar name ‘NCHA3’. This new hydrangea was developed through a breeding program at North Carolina State University, Mills River, N.C. ‘NCHA3’ was selected from a population of seedlings that resulted from a cross of Hydrangea arborescens H2009-068-007 (unpatented) and Hydrangea arborescens H2008-143-001 (unpatented). See FIG. 1 for complete pedigree. The first asexual propagation of ‘NCHA3’ was carried out in July 2012 by rooting stem cuttings at the North Carolina State University, Mountain Horticultural Crops Research Station, Mills River, N.C. and has been asexually reproduced repeatedly by vegetative cuttings over a three year period. ‘NCHA3’ roots readily from softwood cuttings treated with a basal dip of 2,500-5,000 ppm indole butyric acid (potassium salt) in water. ‘NCHA3’ has been found to retain its distinctive characteristics through successive asexual propagations.

### SUMMARY OF THE INVENTION

The following are the unique combination of characteristics of this new cultivar when grown under standard horticultural practices at North Carolina State University, Mountain Horticultural Crops Research Station, Mills River, N.C.

1. Large mop-head inflorescences (i.e., a hortensia flower form) with prevalent sterile flowers.

2. Red flower buds that open with dark pink sepals.

3. Triploid cytotype.

4. Compact form.

### COMPARISON WITH COMMERCIAL CULTIVARS

Table 1 shows distinguishing characteristics between ‘NCHA3’ and other commercially available cultivars of H. arborescens. Hydrangea arborescens ‘NCHA3’ has a unique combination of traits including a mophead inflorescence, red flower buds that open with dark pink sepals, a very compact form, and a triploid cytotype.

TABLE 1
Comparison to other cultivars.

| | Taxa | | | | |
|---|---|---|---|---|---|
| | H. arbor- | H. arbor- | H. arbor- | H. arbor- | H. arbor- |
| | escens | escens | escens | escens | escens |
| Trait | ‘NCHA1 | ‘NCHA2’ | ‘NCHA3’ | ‘NCHA4’ | ‘PIIHA-I’ |
| Ploidy | Diploid | Triploid | Triploid | Triploid | Unknown |
| level | (2.7 pg) | (3.9 pg) | (3.8 pg) | (3.8 pg) | |
| (genome | | | | | |
| size, pg) | | | | | |
| Growth | Rounded | Rounded | Rounded | Rounded | Upright, |
| habit | | | | | spreading |
| Height | 100 cm × | 80 cm × | 60 cm × | 80 cm × | 90 cm × |
| and | 100 cm | 80 cm | 60 cm | 80 cm | 90 cm |
| width | | | | | |
| Stem | 5 to | Avg. 7 cm | Avg. 6 cm | Avg. 7 cm | About |
| internode | 15 cm | (typical | (typical | (typical | 6 cm |
| length | | range: | range: | range: | |
| | | 3 to | 3 to | 3 to | |
| | | 10 cm) | 8 cm) | 10 cm) | |
| Flowers | | | | | |
| per inflor- | | | | | |
| escence | | | | | |
| Fertile | Approx. | Approx. | Approx. | Approx. | Approx. |
| | 600 | 150 | 150 | 200 | 75 |
| Sterile | Approx. | Approx. | Approx. | Approx. | Approx. |
| | 600 | 300 | 300 | 400 | 300 |
| Corymb | 8-20 cm | Avg. | Avg. | Avg. | Avg. |
| diameter | | 14 cm | 12 cm | 20 cm | 12.5 cm |
| Sepal | | | | | |
| color | | | | | |
| Above | Red-Purple | Red- | Red- | Red- | Close to |
| | (ranges from | Purple | Purple | Purple | Red- |
| | 68D/C to | (65A to | (60C) | (63D to | Purple |
| | N66D/N66C) | 67C) | fading | 65B) | (58A) |
| | | fading | to Red- | | |
| | | to Red- | Purple | | |
| | | Purple | (70C) | | |
| | | (65C) | | | |
| Below | Red-Purple | Red- | Red- | Red- | Close to |
| | (ranges from | Purple | Purple | Purple | Red- |
| | 68D/C to | (63B) | (60A) | (63A) | Purple |
| | N66D/N66C) | | | | (63A) |
| Sepal | | | | | |
| Length | 0.5-0.7 cm | Avg. | Avg. | Avg. | 0.7 cm |
| | | 1.2 cm | 1.0 cm | 1.0 cm | |
| Width | 0.3-0.5 cm | Avg. | Avg. | Avg. | 0.5 cm |
| | | 1.0 cm | 0.7 cm | 0.7 cm | |

### BRIEF DESCRIPTION OF THE DRAWINGS

This new hydrangea is illustrated by the accompanying photographs which show the plant's pedigree, form, foliage and inflorescences. Photographs were taken on 2-year-old plants. The colors shown are as true as can be reasonably obtained by conventional photographic procedures. Colors in the photographs may differ slightly from the color values cited in the detailed botanical description, which accurately describe the colors of the new hydrangea.

FIG. 1 Shows the pedigree of ‘NCHA3’.

FIG. 2 Shows the form of a container-grown plant in flower.

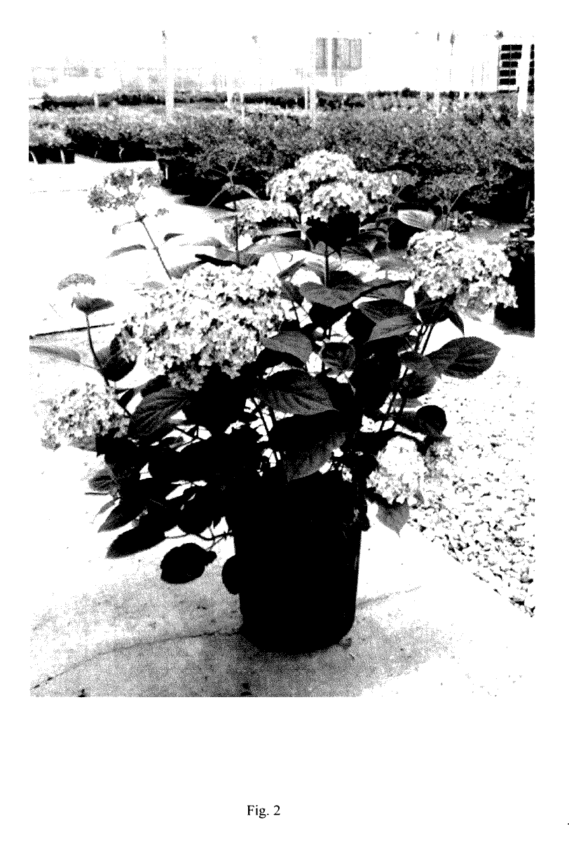

FIG. 3 Shows the form of a field-grown plant in flower.

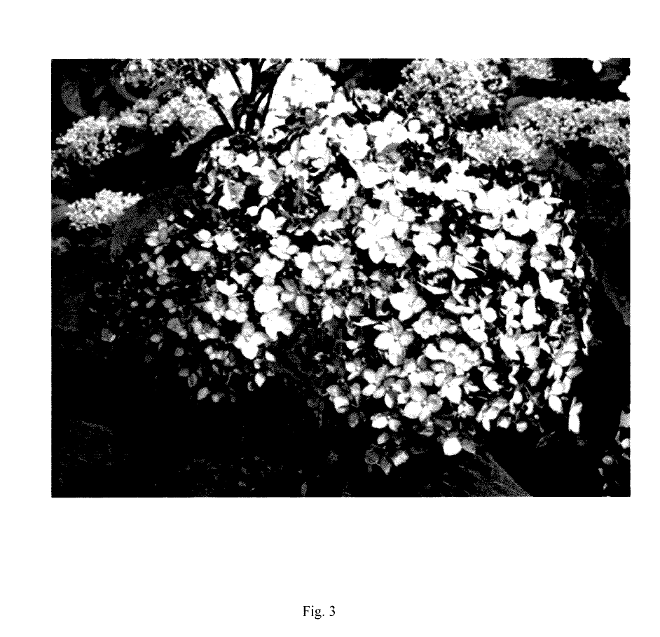

FIG. 4 Shows the deep pink mop-head inflorescence.

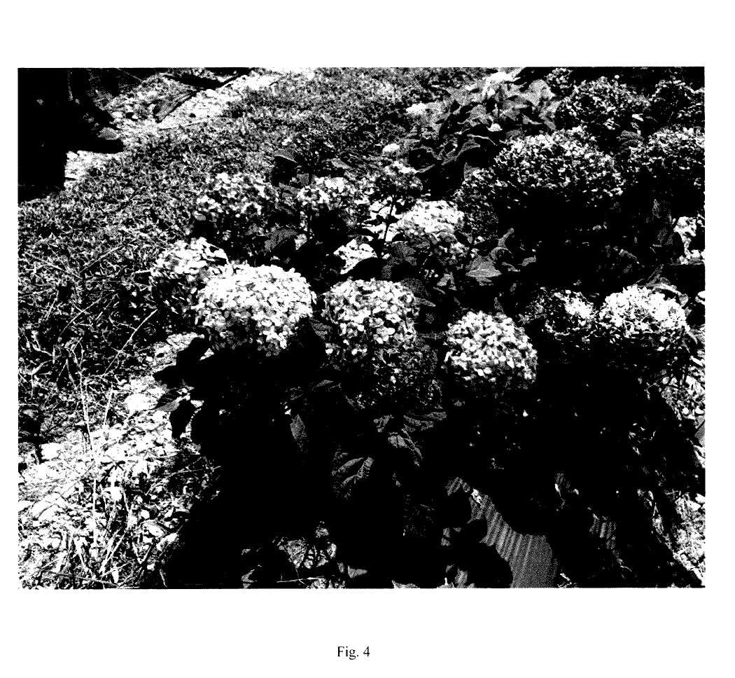

### DETAILED BOTANICAL DESCRIPTION

The following is a detailed description of the botanical characteristics of the new and distinct hydrangea variety plant known by the denomination ‘NCHA3’. The detailed description was taken on a three-year-old field-grown plant in Mills River, N.C. in 2014. All colors cited herein refer to The Royal Horticultural Society Colour Chart (The Royal Horticultural Society (R.H.S.), London, 2001 Edition). Where specific dimensions, sizes, colors, and other characteristics are given, it is to be understood that such characteristics are approximations or averages set forth as accurately as practicable.

### Technical Description of the Variety.

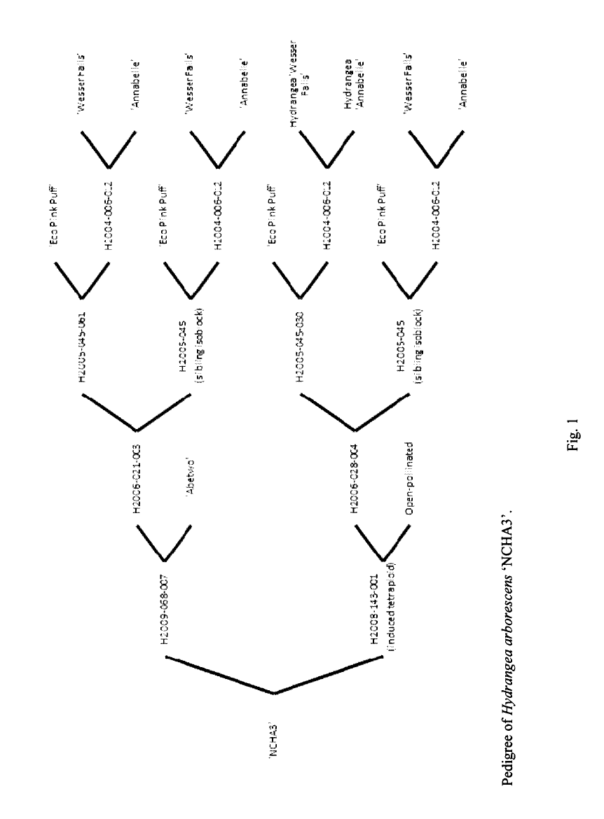

Classification:
   Botanical name.—Hydrangea arborescens ‘NCHA3’.
   Common name.—Smooth Hydrangea.
Parentage: ‘NCHA3’ originated as a seedling that resulted from a cross of H. arborescens H2009-068-007 and H. arborescens H2008-143-001. See FIG. 1 for extended pedigree.
Plant description:
   Growth habit.—Rounded.
   Height (from top of soil including inflorescences).—About 60 cm.
   Width (including inflorescences).—About 60 cm.
Shoot and stem:
   Shoot length.—50 cm (typical range: 40 to 60 cm).
   Shoot diameter.—0.5 cm (typical range: 0.3 to 0.9 cm).
   Internode length.—6 cm (typical range: 3 to 8 cm).
   Shoot texture (current year).—Pubescent.
   Shoot color (current year).—Yellow-Green (146C).
   Mature stem texture (from prior year).—Glabrous.
   Mature stem color (from prior year).—Grey-Brown (199D).
Branching habit: Freely branching with 50-80 shoots.
Leaves:
   Type.—Simple.
   Persistence.—Deciduous.
   Arrangement.—Opposite.
   Shape.—Broadly ovate.
   Apex.—Acuminate.
   Base.—Acute to rounded.
   Venation.—Pinnate, opposite to subopposite.
   Vein color.—Upper: Yellow-Green (148B). Lower: Yellow-Green (148D).
   Margins.—Dentate to serrate.
   Texture.—Upper surface: Glabrous. Lower surface: Slightly pubescent.
   Emerging leaves.—Color: Upper and lower: Yellow-Green (146B).
   Mature leaves during growing season.—Color: Upper: Green (147A). Lower: Yellow-Green (146A to 146B). Length of lamina: Avg. 8 cm (typical range: 5 to 11 cm). Width: Avg. 6 cm (typical range: 4 to 8 cm).
   Leaf attachment.—Petiolate.
   Petiole.—Length: Avg. 3 cm (typical range: 1 to 4 cm). Diameter: Avg. 0.2 cm (typical range: 0.15 to 0.25 cm). Color: Brown (200C to 200D).
Inflorescence:
   Description.—Fertile (approximately 300) and sterile (approximately 150) flowers arranged on individual hemispherical or dome-shaped terminal, compound corymbs.
   Flowering season.—Late May to late June in Mills River, N.C.
   Corymb.—Diameter: Average 12 cm (typical range: 7 to 14 cm). Height: Average 7 cm (typical range: 5 to 8 cm). Quantity: Often 50-80 corymbs per plant over the flowering season. Longevity: Showy for approximately 4 weeks.
   Sterile flowers.—Petals, pistils and stamens are greatly reduced (<0.5 mm) and insignificant. Buds: Shape: Oval. Diameter: 0.2 cm. Color: Red-Purple (59B). Sepals: Number: 2 to 4. Length: Avg. 1.0 cm (typical range: 0.7 to 1.2 cm). Width: Avg. 0.7 cm (typical range: 0.5 to 1.0 cm). Shape: Oval. Apex: Mucronate to rounded. Base: Rounded. Margin: Entire. Texture: Glabrous. Color: Upper: Red-Purple (60C) fading to Red-Purple (70C). Lower: Red-Purple (60A).
   Fertile flowers.—Sepals and petals are greatly reduced (<0.5 mm) and insignificant. Buds: Shape: Round. Diameter: 0.2 cm. Color: Red-Purple (59B). Width: 0.2 cm. Height: 0.2 cm.
Reproductive organs:
   Gynoecium.—Pistil number: 2, fused. Pistil color: White (155B). Pistil length: 0.2 cm. Stigma shape: Blunt. Stigma diameter: 0.025 cm. Style length: 0.1 cm (typical range: 0.095 to 0.109 cm). Ovary shape: Rounded. Ovary diameter: 0.1 cm.
   Androecium.—Stamen number: 10. Anther: Shape: Rounded, fusiform. Length: 0.05 cm. Color: White (158D). Filament: Length: 0.07 cm. Color: White (155B). Amount of pollen: None observed.
Other characteristics:
Fruit/seed set: Seeds are minute, dust-like. Not accurately measured with R.H.S. chart.
Disease and insect resistance: No significant disease or insect pests have been observed.
Cold hardiness: At least USDA zone 6b; testing has not been completed in colder zones.
Genome size: Flow cytometry was conducted to determine DNA content which is directly correlated with ploidy among closely related taxa. Holoploid, 2C DNA content was determined on newly expanded leaf tissue. Nuclei were extracted, stained with 4′, 6-Diamidino-2-phenylindole (DAPI), and analyzed (minimum of 5,000 nuclei per sample) using a flow cytometer (PA-I, Partec, Munster, Germany). Pisum sativum L. ‘Ctirad’, with a known genome size of 8.75 pg was used as an internal standard. 2C DNA content for ‘NCHA3’ was 3.8 pg, approximately 1.5 times the DNA content of a diploid (e.g., ‘NCHA1’), and consistent with being a triploid as would be expected from an interploid cross between diploid and tetraploid parents (see Table 1).

What is claimed is:

1. A new and distinct cultivar of Hydrangea arborescens named ‘NCHA3’ as illustrated and described herein.
